(19) United States
(12) Patent Application Publication
(10) Pub. No.: US 2021/0388310 A1
(43) Pub. Date: Dec. 16, 2021

(54) METHODS AND COMPOSITIONS FOR PRODUCING HOMOKARYOTIC FILAMENTOUS FUNGAL CELLS

(71) Applicant: PERFECT DAY, INC., BERKELEY, CA (US)

(72) Inventors: Timothy Geistlinger, Oakland, CA (US); Brian Darst, Berkeley, CA (US); Timo Schuerg, Berkeley, CA (US); Balakrishnan Ramesh, Berke, CA (US); Wendy Yoder, Davis, CA (US)

(21) Appl. No.: 17/278,427

(22) PCT Filed: Sep. 20, 2019
(86) PCT No.: PCT/US19/52238

§ 371 Date: Mar. 22, 2021

Related U.S. Application Data
(60) Provisional application No. 62/734,245, filed on Sep. 20, 2018.

(51) Int. Cl. C12N 1/14; C12P 1/02

(52) U.S. Cl. CPC C12N 1/14; C12R 2001/66; C12P 1/02

(57) ABSTRACT

Provided are methods and compositions useful for producing filamentous fungal cells and compounds produced by such cells that have utility in a variety of applications. In one aspect, provided herein is a method for producing a SLR from a filamentous fungal cell, wherein the method comprises the steps of: a) growing the filamentous fungal cell in a first medium comprising a first carbon source to obtain an actively growing mycelial culture, and b) replacing the first medium of the actively growing mycelial culture with a second medium comprising a second carbon source to induce production of the SLR; wherein the first carbon source comprises a metabolizable carbon compound, wherein the second carbon source comprises only non-metabolizable carbon compounds, and wherein the second medium comprises no other carbon source than the second carbon source.

### CROSS-REFERENCE TO RELATED APPLICATIONS

This application claims priority from Provisional Application Ser. No. 62/734,245 filed on Sep. 20, 2018, which is hereby incorporated by reference in its entirety.

### FIELD OF THE INVENTION

Provided are methods and compositions useful for producing filamentous fungal cells and compounds produced by such cells that have utility in a variety of applications.

### BACKGROUND OF THE INVENTION

Fungi are attractive options for expressing and producing recombinant proteins with industrial applications at large scale.

The use of yeast in this application is well established. Yeasts are unicellular (i.e., single cell) and uninucleate (i.e., single nucleus per cell) organisms. These two properties played a fundamental role in the development of rapid, small-scale, high-throughput, parallel genetic engineering and screening platforms, which in turn have helped create the understanding of yeast genetics that now enables the synthetic biology industry to produce small molecules, renewable fuels, food supplements, medicines, and biomaterials.

Filamentous fungi offer a theoretical production capacity for such compounds that far exceeds that of yeast. In addition, intron splicing, secretion of recombinant proteins comprising mammalian signal sequences, and glycosylation and other post-translational modifications of recombinant proteins produced in filamentous fungi more closely resemble those found in mammalian cells than is the case when the recombinant proteins are produced in yeast, making filamentous fungi more attractive for the production of recombinant mammalian proteins.

However, compared to yeast, the genetics and tools for genetic manipulation of filamentous fungi are less well established. This is in large part due to the long, filamentous structures (i.e., hyphae) formed by filamentous fungi. Growing by tip extension, these hyphae constitute the main mode of vegetative growth of filamentous fungi. In many filamentous fungal strains, the hyphae of one individual can fuse with other hyphae of the same individual to create a mycelial network. The entangled mycelial networks formed by filamentous fungi in unstirred cultures, and the highly viscous suspension cultures they form in stirred tank bioreactors, make proper agitation, aeration, nutrient diffusion, and mass transfer of the cultures challenging. Moreover, unlike other multicellular organisms in which rigid cell walls prevent movement of cellular organelles between cells, the cell nuclei of many non-coenocytic species of filamentous fungi can move freely throughout the mycelial network through septal pores, with rates of nuclear migration of up to several microns per second. Consequently, filamentous fungi are multi-nucleate. Upon transformation of a filamentous fungal cell, it is therefore likely that the introduced DNA is taken up by only one or a limited number of all nuclei present in the mycelial network, leading to the formation of a heterokaryon (i.e., a multinucleate cell that contains genetically different nuclei), and making reliable genetic manipulation and analysis (e.g., to characterize the effect of a genetic change) difficult.

Homokaryons (i.e., cells that comprise a single nucleus or multiple nuclei comprising identical genomes) of filamentous fungi can be obtained by inducing sporulation of the hyphae, and then isolating single spores (i.e., conidia). Since the conidia can still contain multiple nuclei, homokaryon isolation via conidia is typically repeated multiple times and combined with colony PCR screening (e.g., using primers that span the selective marker and the targeted genomic locus; colonies that produce the correct PCR products for the genetic modification and absence of the wild-type PCR product are considered to be homokaryotic).

Unfortunately, many industrially used filamentous fungal strains are no longer capable of forming conidia (i.e., are sporulation-deficient). Such strains have typically undergone heavy mutagenesis, during which they appear to have lost genetic information required for conidia formation. This appears to be particularly common in high protein production strains (see, for example, Imran et al. (2011) International Journal of the Physical Sciences 6(26): 6179-90).

The only presently known method for obtaining homokaryons of sporulation-deficient filamentous fungal strains requires repeated isolation and re-streaking onto selective media of the tips of hyphae. Similar to conidia, hyphal tips contain lower numbers of or single nuclei, and can be plated and grown on selective media. However, hyphal tips are small and often grow as highly viscous masses that can be difficult or impossible to isolate by the pipette, syringe, acoustic, or fluidic methods used for isolating single cell yeast and bacteria.

The difficulty of obtaining homokaryons of desirable filamentous fungal strains presents a significant obstacle to applying the workflow of genetic manipulation, clonal selection/screening, and clonal purification that is essential to advancing our understanding of filamentous fungi and our ability to use them in industrial applications.

There, therefore, exists a need for new methods for isolating homokaryotic filamentous fungal cells.

### BRIEF DESCRIPTION OF THE FIGURES

FIGS. 1A-C each show light microscopy photographs of a sporulation-deficient Aspergillus niger strain that was induced to produce SLPs. Labels: A=SLPs forming at the tip of hyphae in solution, B=SLPS that have separated from the hyphae, C=SLPs that look similar to yeast cells, and D=hyphae or filament.

FIG. 2 shows a bar graph of nuclei counts per SLP.

### SUMMARY OF THE INVENTION

In one aspect, provided herein is a method for producing a SLP from a filamentous fungal cell, wherein the method comprises the steps of: a) growing the filamentous fungal cell in a first medium comprising a first carbon source to obtain an actively growing mycelial culture, and b) replacing the first medium of the actively growing mycelial culture with a second medium comprising a second carbon source to induce production of the SLP; wherein the first carbon source comprises a metabolizable carbon compound, wherein the second carbon source comprises only non-metabolizable carbon compounds, and wherein the second medium comprises no other carbon source than the second carbon source.

In another aspect, provided herein is a method for producing a homokaryotic derivative of a filamentous fungal cell, wherein the method comprises the steps of: a) producing a SLP from the filamentous fungal cell; b) germinating the SLP to obtain an actively growing mycelial culture; and c) repeating steps a) and b) until a homokaryotic SLP is obtained.

In another aspect, provided herein is a method for producing a genetically modified derivative of a filamentous fungal cell, wherein the method comprises the steps of: a) producing a plurality of SLPs from the filamentous fungal cell; b) distributing the plurality of SLPs into a plurality of chambers; c) genetically modifying the plurality of SLPs to obtain a plurality of genetically modified SLPs; and d) germinating the plurality of genetically modified SLPs under a selective condition to obtain a genetically modified derivative of a filamentous fungal cell.

In another aspect, provided herein is a method for producing a library of derivatives of a filamentous fungal cell comprising a library of recombinant nucleic acids, wherein the method comprises the steps of: a) producing a plurality of SLPs from the filamentous fungal cell; b) distributing the plurality of SLPs into a plurality of chambers; c) germinating the plurality of SLPs to obtain a plurality of actively growing mycelial cultures; d) producing a second plurality of SLPs from the plurality of actively growing mycelial cultures; e) transforming the second plurality of SLPs with a library of heterologous nucleic acids to obtain a library of SLPs comprising the library of heterologous nucleic acids; and f) germinating the library of SLPs under selective conditions to obtain a library of derivatives of a filamentous fungal cell comprising a library of recombinant nucleic acids.

In another aspect, provided herein is a method for growing a filamentous fungal cell comprising the steps of: a) producing a plurality of SLPs from the filamentous fungal cell; b) preparing an inoculum comprising the plurality of SLPs; c) inoculating a medium with the inoculum to obtain a culture, wherein the medium comprises a metabolizable carbon source; and d) incubating the culture.

### DETAILED DESCRIPTION OF THE INVENTION

Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural, unless otherwise indicated herein or clearly contradicted by context.

The terms “about” and “similar to” as used to herein refer to being within an acceptable error range for the particular value as determined by one of ordinary skill in the art, which can depend in part on how the value is measured or determined, or on the limitations of the measurement system.

The term “and/or” as used herein refer to multiple components in combination or exclusive of one another. For example, “x, y, and/or z” can refer to “x” alone, “y” alone, “z” alone, “x, y, and z”, “(x and y) or z”, or “x or y or z”.

The term “carbon source” as used herein refers to a compound that comprises carbon.

The term “cell” as used herein refers not only to the particular subject cell but to the progeny of such cell. Because certain modifications may occur in succeeding generations due to either mutation or environmental influences, such progeny may not, in fact, be identical to the parent cell, but are still included within the scope of the term “cell” as used herein.

The terms “conidium” and “conidiospore” as used herein refer to spherical, vegetative propagules of filamentous fungi that serve the purpose of propagation and endurance of adverse conditions such as water or nutrient deprivation.

The term “essentially free of” as used herein refers to the indicated component being either not detectable in the indicated composition by common analytical methods, or being present in such trace amounts as to not be functional. The term “functional” as used in this context refers to not contributing to properties of the composition comprising the trace amounts of the indicated component, or to not having health-adverse effects upon consumption of the composition comprising the trace amounts of the indicated component.

The term “filamentous fungal cell” as used herein refers to a cell from any filamentous form of the subdivision Eumycota and Oomycota (as defined by Hawksworth et al., In, Ainsworth and Bisby's Dictionary of The Fungi, 8th edition, 1995, CAB International, University Press, Cambridge, UK). A filamentous fungal cell is distinguished from yeast by its hyphal elongation during vegetative growth.

The term “fungus” as used herein refers to organisms of the phyla Ascomycotas, Basidiomycota, Zygomycota, and Chythridiomycota, Oomycota, and Glomeromycota. It is understood, however, that fungal taxonomy is continually evolving, and therefore this specific deliberation of the fungal kingdom may be adjusted in the future.

The term “heterologous” as used herein refers to not being normally present in the context employed. In other words, an entity thus characterized is foreign in the context in which it is described. When used in reference to a protein that is produced by a filamentous fungal cell, the term implies that the protein is not natively produced by the filamentous fungal cell.

The term “identical” as used herein in the context of nucleic acid or protein sequences refers to the residues in the two sequences that are the same when aligned for maximum correspondence. There are a number of different algorithms known in the art that can be used to measure nucleotide sequence or protein sequence identity. For instance, sequences can be compared using FASTA (e.g., using its default parameters as provided in the Wisconsin Package Version 10.0, Genetics Computer Group (GCG), Madison, Wis.), Gap (e.g., using its default parameters as provided in the Wisconsin Package Version 10.0, GCG, Madison, Wis.), Bestfit, ClustalW (e.g., using defaust paramaters of Version 1.83), and BLAST (e.g., using reciprocal BLAST, PSI-BLAST, BLASTP, BLASTN) (see, for example, Pearson. 1990. Methods Enzymol. 183:63; Altschul et al. 1990. J. Mol. Biol. 215:403).

The terms “including,” “includes,” “having,” “has,” “with,” or variants thereof as used herein are intended to be inclusive in a manner similar to the term “comprising”.

The term “metabolizable carbon source” as used herein refers to a carbon source that as sole carbon source is sufficient to support cellular growth of a filamentous fungal cell.

The term “native” as used herein refers to what is found in nature.

The term “non-metabolizable carbon source” as used herein refers to a carbon source that as sole carbon source is insufficient to support cellular growth of a filamentous fungal cell.

The nucleic acids disclosed herein may include both sense and antisense strands of RNA, cDNA, genomic DNA, and synthetic forms and mixed polymers of the above. They may be modified chemically or biochemically or may contain non-natural or derivatized nucleotide bases. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids) Examples of modified nucleotides are known in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The terms “optional” or “optionally” as used herein refer to a feature or structure being present or not, or an event or circumstance occurring or not. The description includes instances in which a particular feature or structure is present and instances in which the feature or structure is absent, or instances in which the event or circumstance occurs and instances in which the event or circumstance does not occur.

The term “protein” as used herein refers to a polymeric form of amino acids of any length, which can include coded and non-coded amino acids, amino acids that occur in nature and those that do not occur in nature, chemically or biochemically modified or derivatized amino acids, and polypeptides having modified peptide backbones.

The term “recombinant” as used herein in reference to a nucleic acid (e.g., a gene) describes a nucleic acid that has been removed from its naturally occurring environment, a nucleic acid that is not associated with all or a portion of a nucleic acid abutting or proximal to the nucleic acid when it is found in nature, a nucleic acid that is operatively linked to a nucleic acid that it is not linked to in nature, or a nucleic acid that does not occur in nature. The term “recombinant” can be used, e.g., to describe cloned DNA isolates, or a nucleic acid including a chemically-synthesized nucleotide analog. A nucleic acid is also considered “recombinant” if it contains any modifications that do not naturally occur to the corresponding nucleic acid in a genome. For instance, an endogenous coding sequence is considered “recombinant” if it contains an insertion, deletion, or a point mutation introduced artificially, e.g., by human intervention. A “recombinant nucleic acid” also includes a nucleic acid integrated into a host cell chromosome at a heterologous site and a nucleic acid construct present as an episome. When “recombinant” is used herein to describe a protein, it refers to a protein that is produced in a cell of a different species or type as compared to the species or type of cell that produces the protein in nature. The term “recombinant filamentous fungal host cell” as used herein refers to a filamentous fungal cell into which a recombinant nucleic acid has been introduced.

The term “yeast” as used herein refers to organisms of the order Saccharomycetales, such as Saccharomyces cerevisiae and Pichia pastoris. Vegetative growth of yeast is by budding/blebbing of a unicellular thallus, and carbon catabolism may be fermentative.

### Method for Producing a Homokaryotic Derivative of Filamentous Fungal Cell

In one aspect, provided herein is a method for producing a homokaryotic derivative of a filamentous fungal cell comprising the step of producing a spore-like propagule (SLP) from the filamentous fungal cell.

The invention is based on the surprising discovery that filamentous fungal cells, including sporulation-deficient filamentous fungal cells, can be induced to produce novel, conidiospore-like entities. The inventors have named such entities spore-like propagules (SLPs).

SLPs are similar to conidia in that they have a spherical, non-filamentous shape; are small (with diameters of between 3 um and 10 um); comprise a small number of nuclei (typically between 1 and 10 nuclei); and can germinate to form another generation of vegetative hyphae (which in turn can give rise to SLPs). Repetitive cycles of the steps of a) producing a SLP, followed by b) germinating the SLP to produce a mycelial culture will lead to production of a homokaryotic SLP that can be germinated to obtain a homokaryotic derivative of a filamentous fungal cell.

The inventors have further discovered that SLPs can be formed by filamentous fungal cells that are genetically engineered to have the ability to form SLPs, as well as by filamentous fungal cells that are not specifically engineered but that are induced to form SLPs (e.g., upon change of carbon source in growth medium).

The inventors have further discovered that SLP formation can continue in culture leading to production of progressively more homokaryotic cells until eventually (e.g., after between 1 and 15 rounds [e.g., between 1 and 15, 14, 13, 12, 11, 10, 9, 8, 7, 6, 5, 4, 3, or 2 rounds; between 2 and 15, 14, 13, 12, 11, 10, 9, 8, 7, 6, 5, 4, or 3 rounds; between 3 and 15, 14, 13, 12, 11, 10, 9, 8, 7, 6, 5, 4 rounds; between 4 and 15, 14, 13, 12, 11, 10, 9, 8, 7, 6, or 5 rounds; between 5 and 15, 14, 13, 12, 11, 10, 9, 8, 7, or 6 rounds; between 6 and 15, 14, 13, 12, 11, 10, 9, 8, or 7 rounds; between 7 and 15, 14, 13, 12, 11, 10, 9, or 8 rounds; between 8 and 15, 14, 13, 12, 11, 10, or 9 rounds; between 9 and 15, 14, 13, 12, 11, or 10 rounds; between 10 and 15, 14, 13, 12, or 11 rounds; between 11 and 15, 14, 13, or 12 rounds; between 12 and 15, 14, or 13 rounds; between 13 and 15, or 14 rounds; or between 14 and 15 rounds] of SLP production followed by germination to mycelial culture) a homokaryotic SLP is obtained. When such “self-purifying” of SLPs is allowed to proceed under selective pressure in a colony on a solid medium or in an agitated liquid culture, homokaryotic cells accumulate on the periphery of the colony or throughout the culture, respectively, and can be easily isolated. This stands in stark contrast to the effort required to obtain homokaryotic cells via hyphal tips, which often requires skilled manual manipulation, cumbersome successive re-streaking, and which can require extended growth periods on selective media. For example, on solid media, homokaryons harboring a selective marker can be obtained via hyphal tips in several months for some species, with multiple manual interventions involving several days of labor at a time; accomplishing the task via SLPs requires less than 2 weeks with little or no manual intervention.

SLPs differ from conidia in that they can be formed by both sporulation-competent and sporulation-deficient filamentous fungal cells; are formed from tips and walls of hyphae (conidia form from differentiated conidiophores); and can be generated in submerged culture (i.e., without air interface; conidia generally form at the aerial surface of mycelial networks on the surface of solid or liquid culture medium).

These properties of SLPs enable use of filamentous fungal cells (particularly of sporulation-deficient filamentous fungal cells) in applications in which their use heretofore ranged from cumbersome to unfeasible.

Such applications include but are not limited to microfluidics and nanotechnology applications in which the use of filamentous fungal cells was previously hampered by the entangled mycelial networks and viscous nature of the cultures they form, which made cell manipulations on a small scale (e.g., micro-scale) impossible, as well as by the cross-contamination danger posed by aerial spores.

Such applications further include but are not limited to high-throughput genetics applications, which require easy separation of populations of genetically modified cells into homokaryotic clones for evaluation of individual genotypes and generation of new strains.

### Producing SLP

A SLP can be produced by exposing a filamentous fungal cell to an agent or condition that induces the formation of a SLP, and/or by introducing into a filamentous fungal cell a genetic modification that enables spontaneous formation of a SLP.

Non-limiting examples of agents that induce formation of a SLP include chemical agents (e.g., specific carbon sources, specific nitrogen sources, specific phosphorus sources, specific sulphur sources, specific ratios of nutrients [e.g., specific carbon to nitrogen ratio], specific selection agents). In some embodiments, the chemical agent is N-acetyl-D-glucosamine

Non-limiting examples of conditions that induce formation of a SLP include cellular stress, mechanical stimuli (e.g., agitation), pH change, heating, and sound (e.g., ultrasound).

A non-limiting example of cellular stress includes stress induced by removal of carbon source. SLP formation can, for example, be induced by first growing a filamentous fungal cell in a first medium that comprises a metabolizable carbon source, and then removing the metabolizable carbon source (e.g., by removing the first medium) and replacing in with one or more non-metabolizable carbon sources (e.g., by adding a second medium comprising one or more non-metabolizable carbon compounds as sole carbon sources). Without wishing to be bound by theory, it is believed that removal of the metabolizable carbon source (and/or replacing the metabolizable carbon source with non-metabolizable carbon sources) induces cellular stress in the filamentous fungal cell to which the filamentous fungal cell responds with a rapid morpho-genetic switch from mycelial growth to SLP formation. Non-limiting examples of metabolizable carbon sources include glucose, fructose, sucrose, xylose, starch, cellulose, dextrin and lactose. Non-limiting examples of non-metabolizable carbon sources include N-acetyl-D-glucosamine In some embodiments, the method for producing a SLP provided herein comprises the steps of growing a filamentous fungal cell in a first medium comprising glucose, followed by growing the filamentous fungal cell in a second medium that is essentially free of glucose or any other metabolizable carbon source and comprises N-acetyl-D-glucosamine. In some such embodiments, the second medium comprises N-acetyl-D-glucosamine as sole carbon source (i.e., comprises no other carbon source but N-acetyl-D-glucosamine)

SLPs can be obtained on or in a medium at any scale and in any format. Non-limiting examples of suitable formats include solid media (e.g., on a culture plate), semi-solid media, liquid media (e.g., in tubes, in flasks, in wells of multi-well plates [e.g., 6-well plates, 12-well plates, 24-well plates, 96-well plates, 384-well plates, 1,536-well plates], in droplets), and emulsion media (e.g., at air and water interfaces, at air and oil interfaces, at oil and water interfaces).

SLP production can be monitored by visual inspection. Alternatively, SLP production can be monitored using colorimetric analysis (SLPs produce a red pigment, as well as comprise or take up dyes that can be detected) or antibody staining (e.g., using antibodies that bind to components of the cell wall of SLPs [e.g., proteins, polysaccharides, glycoproteins]).

The number of SLPs produced can be quantified using a cell counter (e.g., Product#AMQAX1000, ThermoFischer Scientific, Waltham, Mass.). Such quantification can be advantageous when preparing an inoculum for a culture according to a method provided herein.

SLPs can be germinated by exposure to a metabolizable carbon source (e.g., by transferring the SLPs to culture medium comprising a metabolizing carbon source). Upon such exposure, SLPs produce a mycelial culture. The mycelial culture can again be induced to produce a SLP by removal of the metabolizable carbon source and addition of a non-metabolizable carbon source to the culture medium.

SLPs can be, optionally, isolated. Non-limiting methods for isolating SLPs include size selection methods (e.g., membrane filtration with suitable size cutoffs, gradient centrifugation), optical methods (e.g., fluorescence activated cell sorting [FACS], light scattering cytometry), and simply plating dilutions to agar media (e.g., agar plates) or liquid wells in microtiter dishes.

### Filamentous Fungal Cell

The filamentous fungal cell disclosed herein or used in the methods disclosed herein can be from any filamentous fungus strain known in the art or described herein, including holomorphs, teleomorphs, and anamorphs thereof.

Non-limiting examples of filamentous fungal cells include cells from an Acremonium, Aspergillus, Aureobasidium, Canariomyces, Chaetonium, Chaetomidium, Corynascus, Cryptococcus, Chrysosporium, Coonemeria, Dactylomyces, Emericella, Filibasidium, Fusarium, Gibberella, Humicola, Magnaporthe, Malbranchium, Melanocarpus, Mucor, Myceliophthora, Myrothecium, Neocallimastix, Neurospora, Paecilomyces, Penicillium, Piromyces, Rhizopus, Schizophyllum, Scytalidium, Sporotrichum, Talaromyces, Thermoascus, Thermomyces, Thielavia, Tolypocladium, or Trichoderma strain.

Non-limiting examples of Acremonium strains include Acremonium alabamense.

Non-limiting examples of Aspergillus strains include Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus niger var. awamori, Aspergillus oryzae, Aspergillus sojae, and Aspergillus terreus, as well as Emericella, Neosartorya, and Petromyces species.

Non-limiting examples of Chrysosporium stains include Chrysosporium botryoides, Chrysosporium carmichaeli, Chrysosporium crassitunicatum, Chrysosporium europae, Chrysosporium evolceannui, Chrysosporium farinicola, Chrysosporium fastidium, Chrysosporium filiforme, Chrysosporium georgiae, Chrysosporium globiferum, Chrysosporium globiferum var. articulatum, Chrysosporium globiferum var. niveum, Chrysosporium hirundo, Chrysosporium hispanicum, Chrysosporium holmii, Chrysosporium indicum, Chrysosporium iops, Chrysosporium keratinophilum, Chrysosporium kreiselii, Chrysosporium kuzurovianum, Chrysosporium lignorum, Chrysosporium obatum, Chrysosporium lucknowense, Chrysosporium lucknowense Garg 27K, Chrysosporium medium, Chrysosporium medium var. spissescens, Chrysosporium mephiticum, Chrysosporium merdarium, Chrysosporium merdarium var. roseum, Chrysosporium minor, Chrysosporium pannicola, Chrysosporium parvum, Chrysosporium parvum var. crescens, Chrysosporium pilosum, Chrysosporium pseudomerdarium, Chrysosporium pyriformis, Chrysosporium queenslandicum, Chrysosporium sigleri, Chrysosporium sulfureum, Chrysosporium synchronum, Chrysosporium tropicum, Chrysosporium undulatum, Chrysosporium vallenarense, Chrysosporium vespertilium, and Chrysosporium zonatum.

Non-limiting examples of Fusarium strains include Fusarium moniliforme, Fusarium venenatum, Fusarium oxysporum, Fusarium graminearum, Fusarium proliferatum, Fusarium verticiollioides, Fusarium culmorum, Fusarium crookwellense, Fusarium poae, Fusarium sporotrichioides, Fusarium sambuccinum, Fusarium torulosum, and associated Gibberella teleomorphs.

Non-limiting examples of Mucor strains include Mucor miehei Cooney et Emerson (Rhizomucor miehei [Cooney & R. Emerson]) Schipper, and Mucor pusillus Lindt.

Non-limiting examples of Myceliophthora strains include Myceliophthora thermophilae.

Non-limiting examples of Neurospora strains include Neurospora crassa.

Non-limiting examples of Penicillium strains include Penicillium chrysogenum and Penicillium roquefortii.

Non-limiting examples of Rhizopus strains include Rhizopus niveus.

Non-limiting examples of Sporotrichum strains include Sporotrichum cellulophilum.

Non-limiting examples of Thielavia strains include Thielavia terrestris.

Non-limiting examples of Trichoderma strains include Trichoderma harzianum, Trichoderma koningii, Trichoderma longibrachiatum, Trichoderma reesei, Trichoderma atroviride, Trichoderma vixens, Trichoderma viride, and alternative sexual/teleomorphic forms thereof (i.e., Hypocrea species).

The filamentous fungal cell may be derived from a wild-type filamentous fungal cell or from a genetic variant (e.g., mutant) thereof.

The filamentous fungal cell may be sporulation-competent or sporulation-deficient. In some embodiments, the filamentous fungal cell is sporulation-deficient.

In some embodiments, the filamentous fungal cell is from a generally recognized as safe (GRAS) industrial stain.

In some embodiments, the filamentous fungal cell has a high exogenous secreted protein/biomass ratio. In some such embodiments, the ratio is greater than about 1:1, greater than about 2:1, greater than about 3:1, greater than about 4:1, greater than about 5:1, greater than about 6:1, greater than about 7:1, or greater than about 8:1. Such high ratios are advantageous in a high-throughput screening environment because they can permit more sensitive and/or rapid screening for secreted proteins.

In some embodiments, the filamentous fungal cell has reduced or eliminated activity of a protease so as to minimize degradation of any protein of interest (see, for example, PCT application WO 96/29391). Filamentous fungal cells with reduced or eliminated activity of a protease can be obtained by screening of mutants or by specific genetic modification as per methods known in the art.

In some embodiments, the filamentous fungal cell is particularly suitable for the high-throughput and/or automated methods and systems provided herein. Non-limiting examples of such filamentous fungal cells include filamentous fungal cells that provide high efficiencies of taking up polynucleotides (e.g., by at least one of the transformation methods provided herein), provide SLPs with a lower number of nuclei or with single nuclei, grow efficiently in microtiter plates, grow faster, produce cultures of lower viscosity (e.g., produce hyphae in culture that are less entangled), have reduced random integration of heterologous polynucleotides (e.g., are inefficient in non-homologous end joining pathway), have increased targeted integration of heterologous polynucleotides (e.g., are efficient in homologous recombination), lack a selectable marker gene, permit use of easily-selectable markers, are efficient and/or accurate at intron splicing, provide high efficiencies of mammalian type post-translational modifications, accept a variety of expression regulatory elements (for ease of use and versatility), permit screening for a wide variety of protein activities or properties, are amenable to ready isolation of the heterologous DNA, and combinations thereof.

### Additional Steps

In some embodiments, the step of obtaining a SLP from a filamentous fungal cell can be combined with additional steps or combinations of additional steps, including steps and combination of steps that are employed when working with unicellular organisms such as bacteria and yeast. Non-limiting examples of such additional steps include: a) one or more additional steps of obtaining a SLP from a filamentous fungal cell; b) genetically modifying a SLP to obtain a genetically modified SLP; c) transforming a SLP to obtain a SLP comprising a recombinant nucleic acid; d) selecting and/or counter-selecting a SLP to obtain a SLP comprising a desired marker; e) screening a SLP to obtain a SLP comprising a desired property; f) inoculating cultures with a SLP; g) growing a SLP; h) germinating a SLP to form hyphae; i) analyzing a SLP; and j) storing a SLP.

### Genetically Modifying SLPs

In some embodiments, SLPs can be genetically modified by random mutagenesis or by site-directed mutagenesis. Random mutagenesis can be accomplished, for example, by chemical mutagenesis (using, for example, ethyl methanesulfonate [EMS], N-methyl-N′-nitro-N-nitrosoguanidine [NTG]), electromagnetic radiation (using, for example, gamma rays, x-rays, UV light), particle radiation (using, for example, fast neutrons, thermal neutrons, beta particles, alpha particles), mock transformation (for example by taking the SLP through the transformation procedure without DNA), and/or random integration of a recombinant nucleic acid.

Site-directed mutagenesis can be accomplished by deleting, substituting, adding, or inverting one or more nucleotides at a specific site in the genome (e.g., by introducing a recombinant nucleic acid in a control sequence that drives expression of a protein or in a coding sequence for a protein to alter expression and/or activity levels of the protein).

Genetic modifications can be introduced using standard genetic engineering techniques (i.e., recombinant technology), classical microbiological techniques, or a combination of such techniques. Some of such techniques are generally disclosed, for example, in Sambrook et al, 1989, Molecular Cloning: A Laboratory Manual, Cold Spring Harbor Labs Press.

### Transforming SLPs

SLPs can be transformed using any method known in the art for transforming filamentous fungal cells. Non-limiting examples of such methods include electroporation, protoplast-mediated transformation (see, for example, Penttila et al. (1987) Gene 61:155-64; Fincham (1989) Microbiol Rev 53:148-70), Agrobacterium-mediated transformation (see, for example, Michielse et al. (2005) Curr Genet 48:1-17), biolistic transformation (see, for example, Ruiz-Diez (2002) J Appl Microbiol 92:189-9), magneto-biolistic transformation, shock-wave-mediated transformation, CaCl2 transformation, and polyethylene glycol (PEG) transformation.

### Selecting and/or Counter-Selecting SLPs

Selecting and counter-selecting enables identification of SLPs (or filamentous fungal cells) that comprise a desired marker, the presence of which indicates successful transformation with a recombinant nucleic acid. Suitable markers for selecting and counter-selecting are known in the art, and include but are not limited to hph (hygromycin phosphotransferase), pat (phosphinothricin acetyl transferase), amdS (acetamimdase), and pyr4 (orotodine 5′ phosphate decarboxylase).

### Screening SLPs

SLPs can be screened for any phenotype that is desired. Non-limiting examples of desired phenotypes include production of a desired compound (e.g., protein, metabolite, small molecule, carbohydrate, lipid, polynucleotide) or of a property or activity associated with such compound (e.g., ability to catalyze a certain chemical reaction, ability to degrade a protein, ability to modify a protein), secretion of a desired compound or of a property or activity associated with such compound; production or secretion of a desired level of a desired compound or of a property or activity associated with such compound; desired cell growth rate; a desired tolerance to a physical or chemical challenge (e.g., heat, pH, agitation, oxygenation, nutrient content in medium); and any other physical, physicochemical, chemical, biological, or catalytic property or any improvement, increase, or decrease in such property.

In some embodiments, screening can be accomplished using methods and technologies known in the art. Non-limiting examples of such methods and technologies include fluorescence assays (e.g., FACS, fluorescent microscopy), colorimetric assays, enzyme reaction assays, polynucleotide hybridization assays, microscopy, flow cytometry, and combinations thereof.

Screening can be performed in any format (e.g., in solution, in plates, on solid medium, in microarrays) and at any scale (e.g., low-throughput, medium-throughput, high-throughput).

For embodiments in which screening aims to detect production and/or secretion of a desired protein, or of amount of production and/or secretion of a desired protein, screening can employ a probe that selectively or specifically binds to the desired protein. Suitable probes are known in the art or can be identified by screening libraries of probes for binding to the desired protein. Non-limiting examples of suitable probes include 8-anilino-1-naphthalenesulfonic acid (ANS; see, for example, Gasymov & Glasgow (2007) Biochim Biophys Acta 1774(3):403-11), retinoic acid (see, for example, Zsila et al. (2002) Biochem Pharmacol 64(11):1651-60), hydrophilic small molecule moieties, and hydrophobic small molecule moieties. In some embodiments, the probes are linked to photo reactive or fluorescent molecules.

For screening in the context of microfluidics applications a suitable probe may be a probe that is linked directly or through a linker molecule to a larger molecule. Such linking can reduce the flow rate of the probe compared to its rate of diffusion in a given medium, allowing local concentrations to increase in a manner that improves localized detection, quantification, and isolation. It can also improve or enable screening using chromatographic methods (e.g., by selectively or specifically binding to a chromatographic support SLPs or filamentous fungal cells that express a specific surface marker). Non-limiting examples of large molecules include beads and other particles (e.g., molecular beads, magnetic beads, charged particles), proteins (e.g., proteins or protein domains that can bind maltose, proteins or protein domains that can bind cellulose, proteins or protein domains that can bind DNA, antibodies), polysaccharides (e.g., cellulose), polynucleotides (e.g., DNA, RNA), polymers (e.g., polyacrylamide), solid substrates (e.g., membranes, chromatographic supports), and other structures (e.g., droplets). Probes are typically attached to such larger molecules via linkers.

### Growing SLPs

SLPs can be grown in any suitable culture medium, in any suitable culture vessel, at any suitable scale, and under any suitable culture condition in which the SLPs provided herein can grow and/or remain viable.

In some embodiments, a suitable culture medium typically comprises carbon, nitrogen (e.g., anhydrous ammonia, ammonium sulfate, ammonium nitrate, diammonium phosphate, monoammonium phosphate, ammonium polyphosphate, sodium nitrate, urea, peptone, protein hydrolysates, yeast extract), and phosphate sources. A suitable culture medium can further comprise salts, minerals, metals, transition metals, vitamins, other nutrients, emulsifying oils, and surfactants. Non-limiting examples of suitable carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof. In some embodiments, the culture media further comprise proteases (e.g., plant-based proteases) that can prevent degradation of the recombinant proteins, protease inhibitors that reduce the activity of proteases that can degrade the recombinant proteins, and/or sacrificial proteins that siphon away protease activity. In some embodiments, the culture medium comprises N-acetyl-D-glucosamine In some such embodiments, the culture medium comprises N-acetyl-D-glucosamine at a concentration of between 0.1% and 20%, 15%, 10%, 8%, 6%, 4%, 2%, or 1%; between 1% and 20%, 15%, 10%, 8%, 6%, 4%, or 2%; between 2% and 20%, 15%, 10%, 8%, 6%, or 4%; between 4% and 20%, 15%, 10%, 8%, or 6%; between 0.1% and 20%, 15%, 10%, or 8%; between 8% and 20%, 15%, or 10%; between 10% and 20%, or 15%; or between 15% and 20%. In some such embodiments, the culture medium comprises N-acetyl-D-glucosamine as the sole carbon source. In some embodiments, the culture medium further comprises 1M sorbitol.

Non-limiting examples of suitable culture vessels include microfluidics chambers, microtiter plates, lab-on-a-chips, microreactors, organ-on-chips, shake flasks, bags (e.g., wave bags), rotary cell culture systems, and fermentors (e.g., stirred tank fermentor, airlift fermentor, bubble column fermentor, fixed bed bioreactor, gas separation membrane bioreactor, continuous bioreactors, scaffold use bioreactor, fluidized bed bioreactor, or any combination thereof).

Suitable culture conditions typically include a suitable pH, a suitable temperature, and a suitable oxygenation.

### Germinating SLPs

SLPs can be germinated to form hyphae by removal of an agent or condition that induces formation of SLPs. In some embodiments, SLPs are germinated in rich medium with a metabolic ale carbon source (i.e., a medium comprising glucose, nitrogen [e.g., yeast extract], essential salts, and trace elements) that is essentially free of N-acetyl-D-glucosamine

### Analyzing SLPs

SLPs can be analyzed by any standard molecular or biochemical method known in the art. Non-limiting examples of such methods include flow cytometry, FACS, fluorescence in situ hybridization (FISH), southern blotting, northern blotting, western blotting, chromatin immunoprecipitation (ChIP), microarray profiling, poly acrylamide gel electrophoresis (PAGE), GC-MS, LC-MS, matrix assisted laser desorption ionization (MALDI), DNA sequencing, RNA sequencing, PCR analysis, whole genome bisulphite sequencing (WGBS), SNP analysis, transcript analysis, genetic stability evaluation, and genetic drift evaluation.

### Storing SLPs

SLPs can be stored by mixing with a cryoprotectant followed by controlled rate cooling of the mixture. Non-limiting examples of suitable cryoprotectants include glycols (e.g., ethylene glycol, propylene glycol, polypropylene glycol [PEG], glycerol, and combinations thereof], dimethyl sulfoxide (DMSO), polyols (propane-1,2-diol, propane-1,3-diol, 1,1,1-tris-(hydroxymethyl)ethane [THME], and 2-ethyl-2-(hydroxymethyl)-propane-1,3-diol [EHMP], and combinations thereof), sugars (e.g., trehalose, sucrose, glucose, raffinose, dextrose, and combinations thereof), 2-Methyi-2,4-pentanedioi (MPD), polyvinylpyrrolidone (PVP), methylcellulose, C-linked antifreeze glycoproteins (C-AFGP), and combinations thereof.

The mixture can be distributed prior to storage to individual cryovial tubes or microtiter plates (e.g., 6-well, 12-well, 24-well, 96-well, 384-well, or 1536-well plate).

The mixture can be stored at any temperature suitable for long-term storage (e.g., −80C, −140C), and can be stored for any duration (e.g., at least 1, 2, 3, 4, 5, 6, 7, 8, 9, 10, 11, 12, or 24 hours; at least 1, 7, 14, 30, or more days; at least 3, 6, 12, or more months).

### Method for Producing Genetically Modified Derivative of Filamentous Fungal Cell

In another aspect, provided herein is a method for producing a genetically modified derivative of a filamentous fungal cell. The method comprises the steps of: a) producing a plurality of SLPs from the filamentous fungal cell; b) distributing the plurality of SLPs into a plurality of chambers; c) genetically modifying the plurality of SLPs to obtain a plurality of genetically modified SLPs; and d) germinating the plurality of genetically modified SLPs under a selective condition (e.g., condition that selects for the presence of a desired trait [e.g., a desired genetic modification] or counter-selects for the presence of an undesired trait [e.g., a lack of correction of a trait]) to obtain a genetically modified derivative of a filamentous fungal cell.

In some embodiments, some of the plurality of SLPs produced from the filamentous fungal cell are homokaryotic. In some embodiments, all of the plurality of SLPs produced from the filamentous fungal cell are homokaryotic.

The distributing of SLPs into chambers and isolating of SLPs from chambers can be accomplished using methods known in the art. Non-liming examples of such methods include cell sorting (e.g., using optically-detectable markers such as a green fluorescent protein that is produced by the SLPs; see, for example, Delgado-Ramos et al. (2014) G3 4(11):2271-78), robotic colony picking, acoustic fluid ejection, and localized dielectrophoresis (DEP) force manipulation (see, for example, U.S. patent publication No. 20170354969).

Non-limiting examples of suitable chambers include wells of a commercially available microtiter plate, gel encapsulated microparticles (GEMs), drops, acoustically ejected fluids (see, for example, U.S. Pat. No. 9,221,250), nano-scale chambers, and pico-scale chambers. In some embodiments, the volume of the chambers is smaller than 1 mL, smaller than 750 uL, smaller than 500 uL, smaller than 250 uL, smaller than 100 uL, smaller than 75 uL, smaller than 50 uL, smaller than 25 uL, smaller than 10 uL, smaller than 5 uL, smaller than 1 uL, smaller than 750 nL, smaller than 500 nL, smaller than 250 nL, smaller than 100 nL, smaller than 75 nL, smaller than 50 nL, smaller than 25 nL, smaller than 10 nL, or smaller than 5 nL. In some embodiments, the volume of the chambers is between 0.5 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, 75 nL, 50 nL, 25 nL, 10 nL, 5 nL, 2.5 nL, or 1 nL; between 1 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, 75 nL, 50 nL, 25 nL, 10 nL, 5 nL, or 2.5 nL; between 2.5 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, 75 nL, 50 nL, 25 nL, 10 nL, or 5 nL; between 5 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, 75 nL, 50 nL, 25 nL, or 10 nL; between 10 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, 75 nL, 50 nL, or 25 nL; between 25 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, 75 nL, or 50 nL; between 50 nL and 1 mL, 750 uL, 500 uL, 250 uL, 100 uL, 75 uL, 50 uL, 25 uL, 10 uL, 5 uL, 1 uL, 750 nL, 500 nL, 250 nL, 100 nL, or 75 nL; between 75 nL and 1 mL, 750 uL, 500 uL, 250 uL, 100 uL, 75 uL, 50 uL, 25 uL, 10 uL, 5 uL, 1 uL, 750 nL, 500 nL, 250 nL, or 100 nL; between 100 nL and 1 mL, 750 uL, 500 uL, 250 uL, 100 uL, 75 uL, 50 uL, 25 uL, 10 uL, 5 uL, 1 uL, 750 nL, 500 nL, or 250 nL; between 250 nL and 1 mL, 750 uL, 500 uL, 250 uL, 100 uL, 75 uL, 50 uL, 25 uL, 10 uL, 5 uL, 1 uL, 750 nL, or 500 nL; between 500 nL and 1 mL, 750 uL, 500 uL, 250 uL, 100 uL, 75 uL, 50 uL, 25 uL, 10 uL, 5 uL, 1 uL, or 750 nL; between 750 nL and 1 mL, 750 uL, 500 uL, 250 uL, 100 uL, 75 uL, 50 uL, 25 uL, 10 uL, 5 uL, or 1 uL; between 1 uL and 1 mL, 750 uL, 500 uL, 250 uL, 100 uL, 75 uL, 50 uL, 25 uL, 10 uL, or 5 uL; between 5 uL and 1 mL, 750 uL, 500 uL, 250 uL, 100 uL, 75 uL, 50 uL, 25 uL, or 10 uL; between 10 uL and 1 mL, 750 uL, 500 uL, 250 uL, 100 uL, 75 uL, 50 uL, or 25 uL; between 25 uL and 1 mL, 750 uL, 500 uL, 250 uL, 100 uL, 75 uL, or 50 uL; between 50 uL and 1 mL, 750 uL, 500 uL, 250 uL, 100 uL, or 75 uL; between 75 uL and 1 mL, 750 uL, 500 uL, 250 uL, or 100 uL; between 100 uL and 1 mL, 750 uL, 500 uL, or 250 uL; between 250 uL and 1 mL, 750 uL, or 500 uL; between 500 uL and 1 mL, or 750 uL; or between 750 uL and 1 mL.

### Method for Producing Library of Derivatives of Filamentous Fungal Cell Comprising Library of Recombinant Nucleic Acids

In another aspect, provided herein is a method for producing a library of derivatives of a filamentous fungal cell comprising a library of recombinant nucleic acids. The method comprises the steps of: a) producing a plurality of SLPs from the filamentous fungal cell; b) distributing the plurality of SLPs into a plurality of chambers; c) germinating the plurality of SLPs to obtain a plurality of actively growing mycelial cultures; d) producing a second plurality of SLPs from the plurality of actively growing mycelial cultures; e) transforming the second plurality of SLPs with a library of heterologous nucleic acids to obtain a library of SLPs comprising the library of heterologous nucleic acids; and f) germinating the library of SLPs under selective conditions to obtain a library of derivatives of a filamentous fungal cell comprising a library of recombinant nucleic acids.

In some embodiments, some of the plurality of SLPs produced from the filamentous fungal cell are homokaryotic. In some embodiments, all of the plurality of SLPs produced from the filamentous fungal cell are homokaryotic.

The method can further comprise the step of screening the library of derivatives of the filamentous fungal cell comprising the library of recombinant nucleic acids to obtain one or more homokaryotic SLPs having a desired phenotype.

### Method for Growing Filamentous Fungal Cell

SLPs provide additional advantages. For example, SLPs can be counted and have high viability, which permits better quantification of culture inocula and prediction of culture growth phase over time than is possible with filamentous fungal cells. SLPs can be propagated without significant mycelial formation in low-viscosity cultures, which greatly facilitates their cultivation in laboratory-scale shaker flasks as well as industrial-scale bioreactors. Low viscosity cultures also allow for better oxygen uptake rates and homogenous culture conditions (e.g., feed rate, pH, salt, surface area), which can facilitate scale-up/scale-down to different fermentation platforms (e.g., platforms run at pico-liter, nanoliter, milliliter, liters, tens of liter, hundreds of liters, thousands of liters, and hundreds of thousands of liters) and which improves biomanufacturing goals and metrics (e.g., productivity, specific productivity, yield, specific yield, titers, and product stability and purification for downstream processing). The low viscosity of SLP cultures also enables continuous bioreactor cultures, which in turn permits the continuous broth removal and the recycling of biomass, improves the down-stream processing (DSP) characteristics of the recombinant compounds according to chemical engineering metrics (including but not limited to reduced flux rates during cell separations, centrifugation separation in disc-stack centrifuges, filtration, rotary vacuum drum filtration (RVDF), plate filtration, dead-end filtration (DEF), chromatography, Ultra-filtration(UF), diafiltration (DF), cross flow filtration, tangential flow filtration (TFF)), and enables better diagnostics during bioreactor performance evaluation than is possible with filamentous fungal cells. Low viscosity also facilitates visualization and pigmentation via fluorescent or color staining, and enables sorting via cytometry (e.g., FACS or cell sorting without fluorescence).

SLPs have a spherical to slightly ovoid morphology, which provides for a larger surface area to volume ratio, and consequently potentially increased yields of secreted compounds (e.g., proteins [e.g., endogenous proteins, recombinant proteins], metabolites, small molecules) than is achieved with mycelial networks of filamentous fungal cells.

Therefore, in yet another aspect, provided herein is a method for growing a filamentous fungal cell comprising the steps of: a) producing a plurality of SLPs from the filamentous fungal cell; b) preparing an inoculum comprising the plurality of SLPs; c) inoculating a medium with the inoculum to obtain a culture, wherein the medium comprises a metabolizable carbon source; and d) incubating the culture.

In some embodiments, some of the plurality of SLPs produced from the filamentous fungal cell are homokaryotic. In some embodiments, all of the plurality of SLPs produced from the filamentous fungal cell are homokaryotic.

In some embodiments, the inoculum comprises a defined number of the plurality of SLPs.

In some embodiments, the culture does not comprise an agent or condition that induces formation of SLPs such that growing the culture occurs by hyphal growth.

In some embodiments, the filamentous fungal cell is capable of producing a protein, and the method comprises the additional step of isolating the protein from the culture.

### Throughput, Scale, Automation

The methods provided herein can be carried out at low-, middle-, or high-throughput. As used herein, the term “high-throughput” refers to the processing of at least 500, at least 1,000, at least 5,000, at least 10,000, at least 50,000, or at least 100,000 samples per day.

The methods provided herein can be carried out at any scale. In some embodiments, the methods provided herein are carried out in a microfluidics device. In some embodiments, the methods provided herein are carried out in a nanofluidics device.

One or more steps of the methods provided herein can be semi-automated or fully automated.

### Filamentous Fungal Cell Capable of Forming SLP

In another aspect, provided herein is a filamentous fungal cell that is capable of forming a SLP. A filamentous fungal cell capable of forming a SLP can be obtained by exposing it to an agent or condition that can induce the formation of a SLP (e.g., medium comprising N-acteyl-D-glucosamine as the sole carbon source) and visually screening for formation of the SLP (using, for example, a dissecting and light microscope).

In some embodiments, the filamentous fungal cell provided herein is capable of forming a SLP comprising a lower number of nuclei (e.g., less than 3) or a single nucleus. Such filamentous fungal cell can be obtained by gel or membrane filtration based on size, as well as FACS using nuclear staining, side scattering, and/or chitin staining.

In some embodiments, the filamentous fungal cell provided herein is capable of forming conidia and a SLP, but forms conidia in response to a different agent or condition than the agent or condition in response to which it forms a SLP.

In some embodiments, the filamentous fungal cell provided herein comprises a recombinant nucleic acid that encodes a recombinant protein. The recombinant protein can be derived from any source. Non-limiting examples of such sources include animals, plants, algae, fungi, and microbes. In some embodiments, the recombinant protein is a recombinant animal protein (i.e., a protein that is natively produced by an animal [e.g., insects (e.g., fly), mammals (e.g., cow, sheep, goat, rabbit, pig, human), birds (e.g., chicken)] or is derived from such a protein [e.g., via insertion, deletion, or substitution of one or more amino acids, or via fragmentation or fusion of such protein]). In some such embodiments, the recombinant animal protein is a protein that comprises a sequence of at least 20 amino acids [e.g., at least 20, at least 30, at least 40, at least 50, at least 60, at least 70, at least 80, at least 90, at least 100, or at least 150, and usually not more than 200 amino acids] that is at least 80% identical [e.g., at least 85%, at least 90%, at least 95% identical, at least 99% identical] to a sequence of amino acids in an animal protein (e.g., a structural protein [e.g., a collagen, a tropoelastin, an elastin], a milk protein [e.g., b-lactalbumin], an egg protein [e.g., ovalalbumin]). In some embodiments, the recombinant protein is a recombinant plant protein (i.e., is derived from a protein that is produced by a plant [e.g., pea, potato]). In some such embodiments, the recombinant plant protein is a protein that comprises a sequence of at least 20 amino acids [e.g., at least 20, at least 30, at least 40, at least 50, at least 60, at least 70, at least 80, at least 90, at least 100, or at least 150, and usually not more than 200 amino acids] that is at least 80% identical [e.g., at least 85%, at least 90%, at least 95% identical, at least 99% identical] to a sequence of amino acids in a plant protein (e.g., a Pisum sativum protein, a potato protein).

In some embodiments, the filamentous fungal cell comprises elevated levels and/or activity of cell division control protein 42 homolog (Cdc42).

In some embodiments, the recombinant nucleic acid encodes a recombinant milk protein. The term “recombinant milk protein” as used herein refers to a milk protein that is produced recombinantly. The term “milk protein” as used herein refers to a protein that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a protein found in a mammal-produced milk.

The recombinant milk protein can be a recombinant whey protein or a recombinant casein. The term “whey protein” or “casein” as used herein refers to a polypeptide that comprises a sequence of at least 20 amino acids (e.g., at least 20, at least 30, at least 40, at least 50, at least 60, at least 70, at least 80, at least 90, at least 100, or at least 150, and usually not more than 200 amino acids) that is at least 80% identical (e.g., at least 85%, at least 90%, at least 95% identical, at least 99% identical) to a sequence of amino acids in a whey protein or casein, respectively. Non-limiting examples of whey proteins include α-lactalbumin, β-lactoglobulin, lactoferrin, transferrin, serum albumin, lactoperoxidase, and glycomacropeptide. Non-limiting examples of caseins include β-casein, γ-casein, κ-casein, α-S1-casein, and α-S2-casein. Non-limiting examples of nucleic acid sequences encoding whey proteins and caseins are disclosed in PCT filing PCT/US2015/046428 filed Aug. 21, 2015, and PCT filing PCT/US2017/48730 filed Aug. 25, 2017, which are hereby incorporated herein in their entireties.

The recombinant milk protein can be derived from any mammalian species, including but not limited to cow, human, sheep, goat, buffalo, camel, horse, donkey, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, and echidna.

The recombinant milk protein can lack epitopes that can elicit immune responses in a human or animal Such recombinant milk proteins are particularly suitable for use in compositions that are edible or ingested (e.g., food products, pharmaceutical formulations, hemostatic products).

The recombinant milk protein can have a post-translational modification. The term “post-translational modification”, or its acronym “PTM”, as used herein refers to the covalent attachment of a chemical group to a protein after protein biosynthesis. PTM can occur on the amino acid side chain of the protein or at its C- or N-termini. Non-limiting examples of PTMs include glycosylation (i.e., covalent attachment to proteins of glycan groups [i.e., monosaccharides, disaccharides, polysaccharides, linear glycans, branched glycans, glycans with galf residues, glycans with sulfate and/or phosphate residues, D-glucose, D-galactose, D-mannose, L-fucose, N-acetyl-D-galactose amine, N-acetyl-D-glucose amine, N-acetyl-D-neuraminic acid, galactofuranose, phosphodiesters, N-acetylglucosamine, N-acetylgalactosamine, sialic acid, and combinations thereof; see, for example, Deshpande et al. 2008. Glycobiology 18(8):6261 via C-linkage, N-linkage, or O-linkage, or via glypiation [i.e., addition of a glycosylphosphatidylinositol anchor] or phosphoglycosylation [i.e., linked through the phosphate of a phospho-serine]), phosphorylation (i.e., covalent attachment to proteins of phosphate groups), alkylation (i.e., covalent attachment to proteins of alkane groups [e.g, methane group in methylation]), and lipidation (i.e., covalent attachment of a lipid group [e.g., isoprenoid group in prenylation and isoprenylation (e.g., farnesol group in farnesylation, geraniol group in geranylation, geranylgeraniol group in geranylgeranylation), fatty acid group in fatty acylation (e.g., myristic acid in myristoylation, palmitic acid in palmitoylation), glycosylphosphatidylinositol anchor in glypiation]), hydroxylation (i.e., covalent attachment of a hydroxide group), sumoylation (i.e., attachment to proteins of Small Ubiquitin-like Modifier (or SUMO) protein), nitrosylation (i.e., attachment to proteins of an NO group), and tyrosine nitration (i.e., attachment to tyrosine residues of proteins of nitrate groups). The PTMs of the recombinant milk protein monomers can be native PTMs, non-native PTMs, or a mixtures of at least one native PTM and at least one non-native PTM. The term “non-native PTM” as used herein refers to a difference in one or more location(s) of one or more PTMs (e.g., glycosylation, phosphorylation) in a protein, and/or a difference in the type of one or more PTMs at one or more location(s) in a protein compared to the native protein (i.e., the protein having “native PTMs”).

The recombinant milk protein can have a milk protein repeat. The term “milk protein repeat” as used herein refers to an amino acid sequence that is at least 80% identical (e.g., at least 85%, at least 90%, at least 95% identical, at least 99% identical) to an amino acid sequence in a protein found in a mammal-produced milk (e.g., a whey protein, a casein) and that is present more than once (e.g., at least 2, at least 3, at least 4, at least 5, at least 10, at least 15, at least 20, at least 30, at least 40, at least 50, at least 75, at least 100, at least 150, or at least 200 times) in the recombinant milk protein monomer. A milk protein repeat may comprise at least 10, at least 20, at least 30, at least 40, at least 50, at least 75, at least 100, or at least 150, and usually not more than 200 amino acids. A milk protein repeat in a recombinant milk protein can be consecutive (i.e., have no intervening amino acid sequences) or non-consecutive (i.e., have intervening amino acid sequences). When present non-consecutively, the intervening amino acid sequence may play a passive role in providing molecular weight without introducing undesirable properties, or may play an active role in providing for particular properties (e.g., solubility, biodegradability, binding to other molecules).

### SLP

In another aspect, provided herein is a SLP. In some embodiments, the SLP comprises between 1 and 10, 9, 8 7, 6, 5, 4, 3, or 2; between 2 and 10, 9, 8 7, 6, 5, 4, or 3; between 3 and 10, 9, 8 7, 6, 5, or 4; between 4 and 10, 9, 8 7, 6, or 5; between 5 and 10, 9, 8 7, or 6; between 6 and 10, 9, 8 or 7; between 7 and 10, 9, or 8; between 8 and 10, or 9; or between 9 and 10. The number of nuclei comprised in an SLP can be determined by the intensity of staining obtained with optically detectable agents that bind to nuclear markers (e.g., propidium iodide).

In some embodiments, the SLP has a diameter of between 3 and 10 um, 9 um, 8 um, 7 um, 6 um, 5 um, or 4 um; between 4 and 10 um, 9 um, 8 um, 7 um, 6 um, or 5 um; between 5 and 10 um, 9 um, 8 um, 7 um, or 6 um; between 6 and 10 um, 9 um, 8 um, or 7 um; between 7 and 10 um, 9 um, or 8 um; between 8 and 10 um, or 9 um; or between 9 and 10 um. The diameter of an SLP can be determined using a light microscope and a microscope slide micrometer in conjunction with software that allows calibration of the micrometer and calculation of the SLPs area and diameter (e.g., Image J).

In some embodiments, the SLP has a diameter of between 2 um and 7 um and comprises 1 or 2 nuclei.

In some embodiments, the SLP has a diameter of between 2 and 12 um and comprises between 1 and 7 nuclei.

In some embodiments, the SLP comprises a cell wall that comprises different components (e.g., proteins, polysaccharides [e.g., a-glucan, b-glucan, chitin, mannan], glycopeptides [see, for example, Lopes et al. (1997) Microbiology 143: 2255-65], melanin) or different levels of such components than the cell wall of a conidium. The presence and levels of components of cell walls can be determined, for example, by using antibody-based detection assays.

In some embodiments, the SLP comprises a cell wall that comprises similar components (e.g., proteins, polysaccharides, glycopeptides, melanin) or similar levels of such components as the cell wall of a hyphae.

In some embodiments, the SLP has a zeta potential that differs from that of a conidium.

In some embodiments, the SLP has a cell wall that has a thickness that differs from that of a conidium. Cell wall thickness can de deduced from the intensity of staining of a cell with agents that bind cell wall components (e.g., Calcofluor white M2R, Solophenyl Flavine 7GFE, 500, Pontamine Fast Scarlet 4B).

In some embodiments, the SLP comprises a recombinant nucleic acid that encodes a recombinant protein (e.g., a recombinant protein disclosed herein).

In some embodiments, the recombinant nucleic acid encodes a recombinant milk protein (e.g., a recombinant milk protein disclosed herein).

### Culture Comprising SLP

In another aspect, provided herein is a culture that comprises a SLP provided herein. In some embodiments, the culture has a viscosity of less than 200 centipoise (cP), less than 150 cP, less than 100 cP, less than 90 cP, less than 80 cP, less than 70 cP, less than 60 cP, less than 50 cP, less than 40 cP, less than 30 cP, less than 20 cP, or less than 10 cP after 48 or more hours of culturing in the presence of adequate nutrients under optimal or near-optimal growth conditions.

The viscosity of a culture can be quantitated by Brookfield rotational viscometry, kinematic viscosity tubes, falling ball viscometers, or cup type viscometers.

It is to be understood that, while the invention has been described in conjunction with certain specific embodiments thereof, the foregoing description is intended to illustrate and not limit the scope of the invention. Other aspects, advantages, and modifications within the scope of the invention will be apparent to those skilled in the art to which the invention pertains.

### EXAMPLES

The following examples are included to illustrate specific embodiments of the invention. The techniques disclosed in the examples represent techniques discovered by the inventors to function well in the practice of the invention; however, those of skill in the art should, in light of the present disclosure, appreciate that many changes can be made in the specific embodiments that are disclosed and still obtain a like or similar result without departing from the spirit and scope of the invention. Therefore, all matter set forth or shown in the examples is to be interpreted as illustrative and not in a limiting sense.

### Example 1: SLP Formation by Sporulation-Deficient Aspergillus niger Strain

A sporulation-deficient Aspergillus niger strain was grown in a medium comprising glucose as a carbon source. The medium was then removed, and replaced with a SLP inducing culture medium (1M sorbitol, minimal base medium +0.1-10% N-acetyl-D-glucosamine as sole carbon source). Cultures were photographed after 4-6 days of growth.

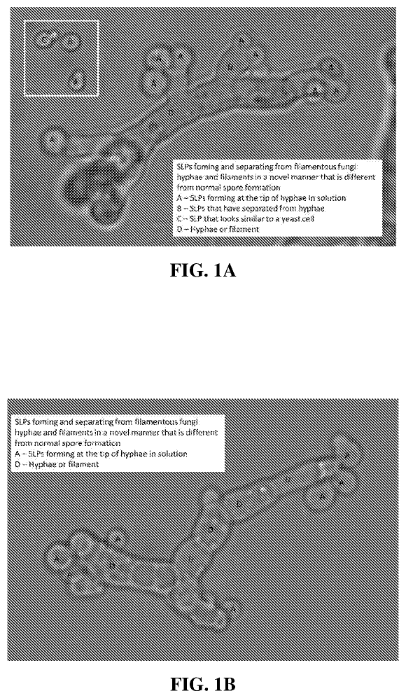

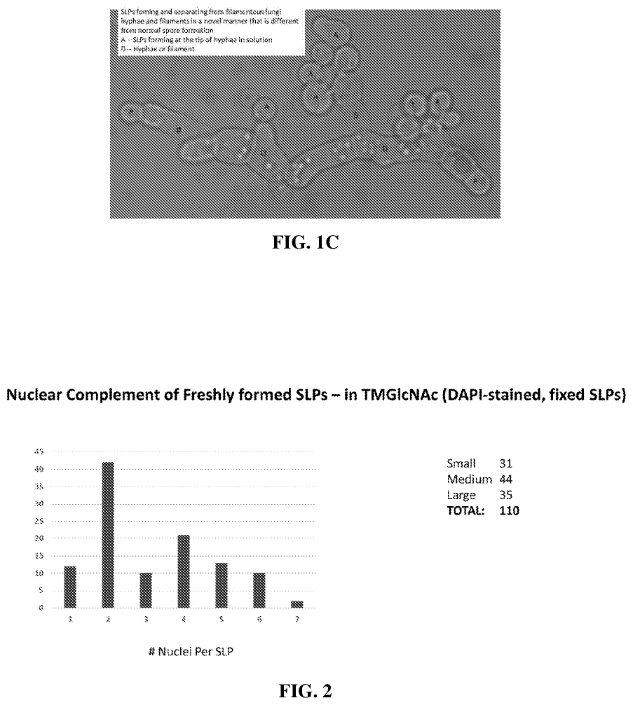

As shown in FIGS. 1A-C, in contrast to conidia, which form from differentiated conidiophores, SLPs are produced directly from the tips and walls of hyphae, under both aerial and submerged conditions. As shown in FIG. 2, the number of nuclei per SLP ranged between 1 and 7.

### Example 2: SLP Inoculum Preparation

A liquid culture of a sporulation-deficient Aspergillus niger strain was grown in a glucose/yeast extract medium, and then stored at −80C or −140C in 25% glycerol. The frozen glycerol mycelial stock was thawed rapidly, and then used to inoculate a shake flask of a relatively rich medium (e.g., Yeast Extract+Glucose). The shake flask culture was incubated at 34C and 200 rpm for 4-6 days (in the light, dark, or uncontrolled light/dark). The 4 day old culture was transferred to one or more sterile 50 ml Falcon tubes, and spun down for 8 minutes at 4,000 rpm at 25C. After decanting the supernatant, the pellet was resuspended in sterile distilled water, mixed thoroughly, and spun again. This washing step was repeated, and the pellet was finally resuspended in an osmotically buffered, minimal medium in which the sole carbon source was N-acetyl-D-glucosamine (GlcNac). The resuspended pellet was used to inoculate either agar plates or shake flasks comprising the above described N-acetyl-D-glucosamine medium. Plates were incubated at 34C for 5-10 days in light, dark, or light/dark.

Once the presence of adequate numbers of SLPs was confirmed microscopically in shake flasks, the SLPs were harvested. To this end, the culture broth was filtered through sterile, triple layered Miracloth (to trap any mycelial fragments) set in a sterile plastic funnel, and the filtrate was collected into sterile 50 mL Falcon tubes, which were then spun down for 6 minutes at 4,000 rom at 25C. The supernatants were decanted, and the pellets containing the SLPs were resuspended in sterile distilled water, mixed and re-spun as above. This washing step was repeated, and the twice-washed pellets were resuspended in the medium of choice, depending on the intended application.

Once the presence of adequate numbers of SLPs was confirmed microscopically on agar plates, the SLPs were harvested. To this end, 5-10 mL of sterile distilled water were added to each plate, and the SLPs were dislodged gently but firmly from the aerial mycelia using a sterile plastic spreader. The SLP-water solution was taken up in a pipet from each plate, and transferred into a triple layer of sterile Miracloth (to trap any mycelial fragments) set in a sterile plastic funnel sitting in a sterile 50 mL Falcon tube. The filtrates containing the SLPs were spun down in the Falcon tube, and the pellets were washed and finally resuspended as described above for the flask process.

All publications, patents, patent applications, sequences, database entries, and other references mentioned herein are incorporated by reference to the same extent as if each individual publication, patent, patent application, sequence, database entry, or other reference was specifically and individually indicated to be incorporated by reference. In case of conflict, the present specification, including definitions, will control. The terminology and description used herein is for the purpose of describing particular embodiments only and is not intended to limit the scope of the invention.

What is claimed is:

1. A method for producing a SLP from a filamentous fungal cell, wherein the method comprises the steps of:
growing the filamentous fungal cell in a first medium comprising a first carbon source to obtain an actively growing mycelial culture; and
replacing the first medium of the actively growing mycelial culture with a second medium comprising a second carbon source to induce production of the SLP, wherein the first carbon source comprises a metabolizable carbon compound, wherein the second carbon source comprises only non-metabolizable carbon compounds, and wherein the second medium comprises no other carbon source than the second carbon source.

2. The method of claim 1, wherein the first carbon source comprises glucose.

3. The method of claim 1, wherein the second carbon source comprises N-acetyl-D-glucosamine

4. The method of claim 1, wherein the first carbon source is glucose and the second carbon source is N-acetyl-D-glucosamine.

5. The method of claim 1, wherein the method further comprises the step of isolating the SLP.

6. The method of claim 1, wherein the filamentous fungal cell is a member of the genus Aspergillus.

7. A method for producing a homokaryotic derivative of a filamentous fungal cell, wherein the method comprises the steps of:
producing a SLP from the filamentous fungal cell;
germinating the SLP to obtain an actively growing mycelial culture; and
repeating steps a) and b) until a homokaryotic SLP is obtained.

8. The method of claim 7, wherein the homokaryotic SLP is germinated to obtain a homokaryotic mycelial culture.

9. The method of claim 7, wherein the filamentous fungal cell is a member of the genus Aspergillus.

10. A method for producing a genetically modified derivative of a filamentous fungal cell, wherein the method comprises the steps of:
producing a plurality of SLPs from the filamentous fungal cell;
distributing the plurality of SLPs into a plurality of chambers;
genetically modifying the plurality of SLPs to obtain a plurality of genetically modified SLPs; and
germinating the plurality of genetically modified SLPs under a selective condition to obtain a genetically modified derivative of a filamentous fungal cell.

11. The method of claim 10, wherein the plurality of SLPs comprises a homokaryotic SLP.

12. The method of claim 10, wherein the plurality of SLPs consists of a plurality of homokaryotic SLPs.

13. The method of claim 10, wherein the filamentous fungal cell is a member of the genus Aspergillus.

14. A method for producing a library of derivatives of a filamentous fungal cell comprising a library of recombinant nucleic acids, wherein the method comprises the steps of:
producing a plurality of SLPs from the filamentous fungal cell;
distributing the plurality of SLPs into a plurality of chambers;
germinating the plurality of SLPs to obtain a plurality of actively growing mycelial cultures;
producing a second plurality of SLPs from the plurality of actively growing mycelial cultures;
transforming the second plurality of SLPs with a library of heterologous nucleic acids to obtain a library of SLPs comprising the library of heterologous nucleic acids; and
germinating the library of SLPs under selective conditions to obtain a library of derivatives of a filamentous fungal cell comprising a library of recombinant nucleic acids.

15. The method of claim 14, wherein the plurality of SLPs comprises a homokaryotic SLP.

16. The method of claim 14, wherein the plurality of SLPs consists of a plurality of homokaryotic SLPs.

17. The method of claim 14, wherein the filamentous fungal cell is a member of the genus Aspergillus.

18. A method for growing a filamentous fungal cell comprising the steps of:
producing a plurality of SLPs from the filamentous fungal cell;
preparing an inoculum comprising the plurality of SLPs;
inoculating a medium with the inoculum to obtain a culture, wherein the medium comprises a metabolizable carbon source; and
incubating the culture.

19. The method of claim 18, wherein the plurality of SLPs comprises a homokaryotic SLP.

20. The method of claim 18, wherein the plurality of SLPs consists of a plurality of homokaryotic SLPs.

21. The method of claim 18, wherein the filamentous fungal cell is a member of the genus Aspergillus.
